(19) United States
(12) Patent Application Publication
(10) Pub. No.: US 2020/0296971 A1
(43) Pub. Date: Sep. 24, 2020

(54) COMPOSITIONS AND METHODS FOR DISPERSING BIOFILMS

(71) Applicant: Texas State University, San Marcos, TX (US)

(72) Inventors: Robert J.C. McLean, Wimberley, TX (US); Sara R. Robertson, Austin, TX (US)

(21) Appl. No.: 16/089,156

(22) PCT Filed: Mar. 31, 2017
(86) PCT No.: PCT/US2017/025306

§ 371 Date: Sep. 27, 2018

Related U.S. Application Data
(60) Provisional application No. 62/316,952, filed on Apr. 1, 2016.

(51) Int. Cl. A01N 59/14; A61L 2/18

(52) U.S. Cl. CPC A01N 59/14; A61L 2/18

(57) ABSTRACT

In some embodiments, a method may include inhibiting and/or dispersing a biofilm. The method may include administering a composition to a biofilm on a surface. In some embodiments, the biofilm may include a plurality of microorganisms coupled together. The composition may include a boric acid. In some embodiments, at least a portion of the composition is dissolved in a solvent. In some embodiments, the method may include dispersing the biofilm using the composition. In some embodiments, the method may include dispersing the biofilm by uncoupling at least some of the plurality of microorganisms. In some embodiments, the method may include administering to the surface an antimicrobial treatment.

### BACKGROUND OF THE INVENTION

### Field of the Invention

The present disclosure generally relates dispersing biofilms. More particularly, the disclosure generally relates to systems and methods for dispersing biofilms using compositions including boric acid.

### 2. DESCRIPTION OF THE RELEVANT ART

Surface-adherent microbial communities (“biofilms”) form when bacteria assemble and adhere to a variety surfaces in, for example, aqueous environments. The bacteria's adherence to surfaces is enabled when they excrete a substance that can anchor them to materials such as metals, plastics, soil particles, biomedical materials and tubing, and even human tissue. Biofilms result in several detrimental conditions in many industries (e.g., fouling and corrosion). Growth within biofilms enables bacteria to persist in spite of treatment with disinfectants and antibiotics.

Biofilms pose numerous problems in a wide variety of industries ranging from medical, environmental, food and beverage and more. In addition to being implicated as a contributor to the growing antimicrobial resistance, biofilms inflict a significant financial burden upon affected industries. In the biomedical field, biofilms are associated with many chronic infections (e.g., medical devices, catheters).

For example and in the case of a naval surface ship, an economic analysis indicated that the primary cost caused by biofouling was due to increased fuel consumption due to increased frictional drag. The overall cost associated with hull fouling for the Navy's present coating, cleaning, and fouling level was estimated to be about $56M per year for the entire DDG-51 class. The cost of industrial biofouling and biocorrosion is estimated at over $200 billion in the US alone. Additionally, microbial biofilms cause other problems such as fouling water filtration units, corrosion of pipelines, and complicate oil and gas extraction operations.

Obviously biofilms have received a lot of research and development attention due to the enormous negative impact they assert on the environment, industry and healthcare. However, the problem remains largely unsolved with no really scalable solution available to make a significant impact. Two strategies that have been investigated to address the biofilms are using inhibition agents to prevent their formation on surfaces, and biofilm dispersal to remove them from surfaces where they persist. Inhibition methods involve treating surfaces with coatings containing agents that prevent cellular adhesion. While this approach has utility, it is only applicable to unaffected surfaces.

Surfaces already contaminated with biofilms must be addressed by mechanical or chemical dispersion methods, and to date there are a variety of limitations associated with current dispersion methods. Some are too costly to scale, while others are too toxic and environmentally disadvantageous.

Major pharmaceutical companies are backing out of antibiotic research which places the search for alternatives to antibiotics on high demand. Historically, removing bacterial biofilms has been done with either biocides or antibiotics. Although they can be effective biofilm dispersion is a novel method of combating biofilms addressing the biofilm itself not the viable cells within. When cells are dispersed the once resistance cells become once again vulnerable when back in a planktonic state. When combined with common antiseptics and antibiotics, the synergistic effect can effectively eliminate biofilms and their infectious counterparts.

Therefore a system and/or method for treating biofilms that are cost effective, scalable and environmentally friendly would be highly desirable.

### SUMMARY

In some embodiments, a method may include inhibiting and/or dispersing a biofilm. The method may include administering a composition to a biofilm on a surface. In some embodiments, the biofilm may include a plurality of microorganisms coupled together. The composition may include a boric acid. In some embodiments, boric acid may include H₃BO₃. In some embodiments, the composition may include a borate salt such as sodium borate (e.g., Na₃BO₃). In some embodiments, at least a portion of the composition is dissolved in a solvent. In some embodiments, the method may include dispersing the biofilm using the composition. In some embodiments, the method may include dispersing the biofilm by uncoupling at least some of the plurality of microorganisms.

In some embodiments, the biofilm is formed by Proteobacteria (e.g., Gram-negative bacteria). In some embodiments, the biofilm is formed by Firmicutes (e.g., Gram-positive bacteria).

In some embodiments, the method may include administering to the surface an antimicrobial treatment. The antimicrobial treatment may include at least one of biocides, surfactants, antibiotics, antiseptics, detergents, chelating agents, virulence factor inhibitors, ultrasonic treatment, radiation treatment, thermal treatment, and mechanical treatment.

In some embodiments, at least a portion of the composition is dissolved in a solvent, and wherein the boric acid has about a 0.1% to about a 5% (w/v) concentration, about a 0.2% to about a 2% (w/v) concentration, or about a 0.3% to about a 0.7% (w/v) concentration. The boric acid may have a 0.5% (w/v) concentration. The boric acid may have a 0.25% to 2% (w/v) concentration. The boric acid may have a 0.4% to 1.1% (w/v) concentration. The boric acid may have a 0.5% to 1.0% (w/v) concentration. In some embodiments, the solvent may include water. In some embodiments, boric acid concentrations above 1.0% (w/v) concentration may result in toxicity.

In some embodiments, the surface comprises at least a portion of a maritime vessel, at least a portion of a medical device, at least a portion of an otic surface of an animal, at least a portion of a petroleum industrial pipeline, or at least a portion of equipment associated with the food industry.

In some embodiments, a method may include inhibiting formation of a biofilm. The method may include administering a composition to a surface. The composition may include a boric acid. The method may include inhibiting formation of a biofilm on the surface using the composition.

### BRIEF DESCRIPTION OF THE DRAWINGS

Advantages of the present invention may become apparent to those skilled in the art with the benefit of the following detailed description of the preferred embodiments and upon reference to the accompanying drawings.

FIG. 1 depicts a diagram of an embodiment of a method for dispersing biofilms.

FIG. 2A depicts a photograph of a microscope slide of river water with biofilms.

FIG. 2B depicts a photograph of a microscope slide of river water with biofilms dispersed after application of a boric acid solution.

FIG. 3 depicts a bar graph summarizing the results of experiments directed towards the ability of Pseudomonas aeruginosa PAO-1 to grow, in various media, in the presence of various concentrations of boric acid.

FIG. 4 depicts a graph summarizing the results of experiments directed towards determining the ability of boric acid to disperse P. aeruginosa biofilms.

FIG. 5 depicts a graph summarizing the results of experiments directed towards determining the ability of boric acid to enhance the antimicrobial action of betadine (poly(vinyl pyrrolidone) iodine) in artificial urine.

FIG. 6 depicts a graph summarizing the results of experiments directed towards determining the ability of boric acid to enhance the antimicrobial action of betadine in artificial urine without using betadine for a control experiment.

FIG. 7 depicts a graph summarizing the results of experiments directed towards determining the ability of boric acid to enhance the antimicrobial action of betadine (poly(vinyl pyrrolidone) iodine) in artificial urine with glucose.

FIG. 8 depicts a graph summarizing the results of experiments directed towards determining the ability of boric acid to enhance the antimicrobial action of betadine in artificial urine with glucose without using betadine for a control experiment.

FIG. 9 depicts a graph summarizing the results of experiments directed towards determining the ability of boric acid to enhance the antimicrobial action of betadine in Davis Minimal Media.

FIG. 10 depicts a graph summarizing the results of experiments directed towards determining the antimicrobial action of boric acid in Davis Minimal Media.

FIG. 11 depicts a graph summarizing the results of experiments directed towards determining the ability of boric acid to enhance the antimicrobial action of betadine in Luria-Bertani broth.

FIG. 12 depicts a graph summarizing the results of experiments directed towards determining the antimicrobial action of boric acid in Luria-Bertani broth.

FIG. 13 depicts a graph summarizing the results of experiments directed towards determining the ability of boric acid to enhance the antimicrobial action of betadine using 5-day old Pseudomonas aeruginosa PAO-1 biofilms grown in artificial urine.

FIG. 14 depicts a graph summarizing the results of experiments directed towards determining the ability of boric acid to enhance the antimicrobial action of betadine using 5-day old Pseudomonas aeruginosa PAO-1 biofilms grown in artificial urine without betadine therefore acting as the control experiment.

While the invention is susceptible to various modifications and alternative forms, specific embodiments thereof are shown by way of example in the drawings and may herein be described in detail. The drawings may not be to scale. It should be understood, however, that the drawings and detailed description thereto are not intended to limit the invention to the particular form disclosed, but on the contrary, the intention is to cover all modifications, equivalents and alternatives falling within the spirit and scope of the present invention as defined by the appended claims.

The headings used herein are for organizational purposes only and are not meant to be used to limit the scope of the description. As used throughout this application, the word “may” is used in a permissive sense (i.e., meaning having the potential to), rather than the mandatory sense (i.e., meaning must). The words “include,” “including,” and “includes” indicate open-ended relationships and therefore mean including, but not limited to. Similarly, the words “have,” “having,” and “has” also indicated open-ended relationships, and thus mean having, but not limited to. The terms “first,” “second,” “third,” and so forth as used herein are used as labels for nouns that they precede, and do not imply any type of ordering (e.g., spatial, temporal, logical, etc.) unless such an ordering is otherwise explicitly indicated. For example, a “third die electrically connected to the module substrate” does not preclude scenarios in which a “fourth die electrically connected to the module substrate” is connected prior to the third die, unless otherwise specified. Similarly, a “second” feature does not require that a “first” feature be implemented prior to the “second” feature, unless otherwise specified.

Various components may be described as “configured to” perform a task or tasks. In such contexts, “configured to” is a broad recitation generally meaning “having structure that” performs the task or tasks during operation. As such, the component can be configured to perform the task even when the component is not currently performing that task (e.g., a set of electrical conductors may be configured to electrically connect a module to another module, even when the two modules are not connected). In some contexts, “configured to” may be a broad recitation of structure generally meaning “having circuitry that” performs the task or tasks during operation. As such, the component can be configured to perform the task even when the component is not currently on. In general, the circuitry that forms the structure corresponding to “configured to” may include hardware circuits.

Various components may be described as performing a task or tasks, for convenience in the description. Such descriptions should be interpreted as including the phrase “configured to.” Reciting a component that is configured to perform one or more tasks is expressly intended not to invoke 35 U.S.C. § 112, paragraph six, interpretation for that component.

The scope of the present disclosure includes any feature or combination of features disclosed herein (either explicitly or implicitly), or any generalization thereof, whether or not it mitigates any or all of the problems addressed herein. Accordingly, new claims may be formulated during prosecution of this application (or an application claiming priority thereto) to any such combination of features. In particular, with reference to the appended claims, features from dependent claims may be combined with those of the independent claims and features from respective independent claims may be combined in any appropriate manner and not merely in the specific combinations enumerated in the appended claims.

It is to be understood the present invention is not limited to particular devices or biological systems, which may, of course, vary. It is also to be understood that the terminology used herein is for the purpose of describing particular embodiments only, and is not intended to be limiting. As used in this specification and the appended claims, the singular forms “a”, “an”, and “the” include singular and plural referents unless the content clearly dictates otherwise. Thus, for example, reference to “a linker” includes one or more linkers.

### DETAILED DESCRIPTION

### Definitions

Unless defined otherwise, all technical and scientific terms used herein have the same meaning as commonly understood by one of ordinary skill in the art.

The term “antimicrobial” as used herein generally refers to a substance capable of destroying or inhibiting the growth of microbes, prevents the development of microbes, and/or inhibits the pathogenic action of microbes as well as viruses, fungi, and bacteria.

The term “biofilm” as used herein generally refers to a thin layer of densely packed microorganisms encapsulated within an aqueous matrix of, for example, proteins, nucleic acids, and/or polysaccharides. Microbes in biofilms tend to flourish on moist surfaces, aggregating and forming colonies.

The term “connected” as used herein generally refers to pieces which may be joined or linked together.

The term “coupled” as used herein generally refers to pieces which may be used operatively with each other, or joined or linked together, with or without one or more intervening members.

The term “directly” as used herein generally refers to one structure in physical contact with another structure, or, when used in reference to a procedure, means that one process affects another process or structure without the involvement of an intermediate step or component.

### EMBODIMENTS

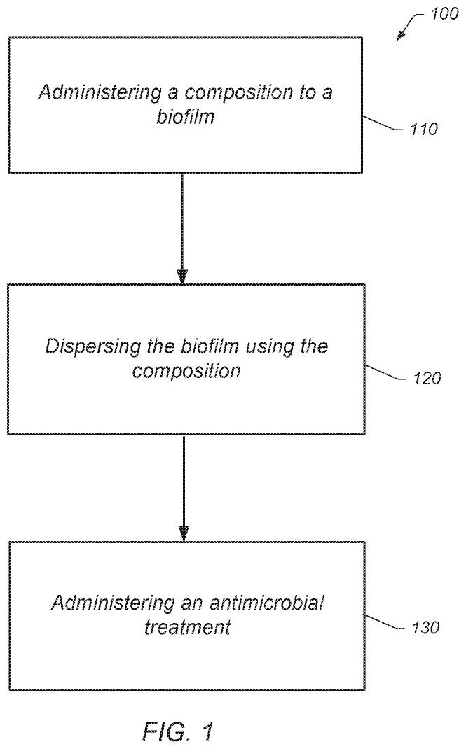

In some embodiments, a method may include inhibiting and/or dispersing a biofilm. FIG. 1 depicts a diagram of an embodiment of a method (100) for dispersing biofilms. The method may include administering a composition to a biofilm on a surface (110). In some embodiments, the biofilm may include a plurality of microorganisms coupled together. The composition may include a boric acid or boric acid derivative (e.g., a salt of a boric acid). In some embodiments, boric acid may include H₃BO₃. For example, boric acid may change to sodium borate (aka borax) at alkaline pH. In some embodiments, at least a portion of the composition is dissolved in a solvent. In some embodiments, the method may include dispersing the biofilm using the composition (120). In some embodiments, the method may include dispersing the biofilm by uncoupling at least some of the plurality of microorganisms.

In some embodiments, at least a portion of the composition is dissolved in a solvent, and wherein the boric acid has about a 0.1% to about a 5% (w/v) concentration. The boric acid may have a 0.5% (w/v) concentration. In some embodiments, the solvent may include a solvent (e.g., lower alcohols, pyridine, acetone) capable of dissolving at least a portion of the boric acid. In some embodiments, the solvent may include a solvent capable of dissolving at least a portion of the boric acid and are non-corrosive and/or environmentally friendly. In some embodiments, the solvent may include an aqueous solvent. In some embodiments, the solvent may include water.

In some embodiments, the method may include administering to the surface an antimicrobial treatment (130). The antimicrobial treatment may include at least one of biocides, surfactants, antibiotics, antiseptics, detergents, chelating agents, virulence factor inhibitors, ultrasonic treatment, radiation treatment, or thermal treatment. In some embodiments, the boric acid treatment and disinfectant treatment steps may be combined (as demonstrated in some of the examples described herein).

An antimicrobial may be generally defined as anything that may kill or inhibit the growth of microbes (e.g., high heat or radiation or a chemical). Microbes may be generally defined as a minute life form, a microorganism, especially a bacterium that causes disease. Antimicrobials may be grouped into three broad categories: antimicrobial drugs, antiseptics, and disinfectants. Antimicrobial drugs may be used in relatively low concentrations in or upon the bodies of organisms to prevent or treat specific bacterial diseases without harming the organism. Unlike antimicrobial drugs, antiseptics and disinfectants are usually nonspecific with respect to their targets, they kill or inhibit a variety of microbes. Antiseptics may be used topically in or on living tissue. Disinfectants may be used on objects or in water.

Antimicrobial resistance may be generally described as a feature of some bacteria that enables them to avoid the effects of antimicrobial agents. Bacteria may possess characteristics that allow them to survive a sudden change in climate, the effects of ultraviolet light from the sun, and/or the presence of an antimicrobial chemical in their environment. Some bacteria are naturally resistant, while other bacteria acquire resistance to antimicrobials to which they once were susceptible.

Biofilms are one example of a characteristic of some microorganisms which allow for certain microorganisms to resist antimicrobials. In some embodiments, compositions described herein may result in dispersal or at least partial dispersal of a biofilm such that known antimicrobial compounds result in better efficacy as regards killing or inhibiting the growth of microbes.

In some embodiments, compositions described herein may include one or more additives. Additives may include, but are not limited to, at least one of biocides, surfactants, antibiotics, antiseptics, detergents, chelating agents, virulence factor inhibitors, gels, polymers, and/or pastes. In some embodiments, the composition may be formulated so that when it is contacted with a biofilm produced by a microorganism, where the biofilm comprises a matrix and microorganism on a surface, the dispersion inducer selectively acts on the microorganism and has a suitable biological response without a required direct effect to disrupt the matrix.

In some embodiments, a method may include inhibiting formation of a biofilm. Compositions discussed herein may be applied to a surface which is prone to formation of biofilms in order to inhibit or prevent formation of the biofilm. The method may include administering a composition (e.g., as discussed herein) to a surface. The composition may include a boric acid. The method may include inhibiting formation of a biofilm on the surface using the composition.

In some embodiments, compositions used to disperse biofilms may be useful in many different industries and environments including, but not limited to: maritime biofouling, biomedical equipment, medical field, veterinary medicine, petroleum industry, and the food industry.

As regards maritime biofouling, portions of a vessel (or associated equipment) sensitive to biofilms can be sensitive to certain antimicrobials. As such the compositions described herein may serve as a non corrosive antimicrobial alternative. Man-made structures such as boat hulls, buoys, drilling platforms, oil production rigs, piers and pipes which are immersed in water are prone to fouling by aquatic organisms. Such structures are commonly of metal, but may include other structural materials such as concrete, wood, synthetic materials, etc. Fouling is a nuisance on boat hulls, because it increases the frictional resistance of the hull's movement through the water, with the consequence of reduced speeds and increased fuel costs. Fowling by aquatic organisms is a nuisance on static structures such as the legs of drilling platforms and oil production rigs, firstly because the resistance of thick layers of fouling to waves and currents can cause unpredictable and potentially dangerous stresses in the structure. Secondly, because fouling makes it difficult to inspect the structure for defects such as stress cracking and corrosion. Fowling by aquatic organisms is a nuisance in pipes such as cooling water intakes and outlets, because the effective cross-sectional area is reduced by fouling, with the consequence of reduced flow rates. Fowling is a nuisance issue as relates to for example tools used in the water, for example nets or fishing rods, especially these items which are left at least partially submerged for long periods of time.

The commercially most successful methods of inhibiting fouling have involved the use of anti-fouling coatings containing substances toxic to aquatic life, for example tributyltin chloride or cuprous oxide. Such coatings, however, are being regarded with increasing disfavor because of the damaging effects such toxins can have if released into the aquatic environment. There is accordingly a need for non-fouling treatments (using methods and compositions described herein) which do not contain markedly toxic materials.

As regards biomedical equipment/medical devices, portions of a device cannot be easily accessed and cleaned by common mechanical scrubbing and as such compositions described herein may be used to clean the device. Medical devices used for patient treatment can be a source of microbial (bacterial or fungal) infection in such patients. For example, insertion or implantation of a catheter into a patient can introduce microbes and/or, when left in place for prolonged periods of time, permit the introduction of microbes during long-term exposure of the catheter exit site to the environment. In addition, long-term catheter use often produces a biofilm on the catheter surface, which facilitates the development of infection that can cause patient discomfort and compromise patient health.

Medical devices are any article that contacts patients or are used in health care, and may be for use either internally or externally. The medical devices can be made from a variety of natural or synthetic materials, such as, for example, latex, polystyrene, polyester, polyvinylchloride, polyurethane, ABS polymers, polyamide, polyimide, polycarbonate, polyacrylates, polyethylene, polypropylene, synthetic rubber, stainless steel, ceramics such as aluminum oxide and glass, and silicone.

Illustrative, non-limiting, examples of medical devices include, but are not limited to, cannulae, catheters, condoms, contact lenses, endotracheal and gastroenteric feeding tubes as well as other tubes, grafts, guide wires, implant devices, IUDs, medical gloves, oxygenator and kidney membranes, pacemaker leads, peristaltic pump chambers, shunts, stents and sutures. Other non-limiting examples of medical devices include peripherally insertable central venous catheters, dialysis catheters, long term tunneled central venous catheters, long term non-tunneled central venous catheters, peripheral venous catheters, short-term central venous catheters, arterial catheters, pulmonary artery Swan-Ganz catheters, urinary catheters, artificial urinary sphincters, long term urinary devices, urinary dilators, urinary stents, other urinary devices, tissue bonding urinary devices, penile prostheses, vascular grafts, vascular catheter ports, vascular dilators, extravascular dilators, vascular stents, extravascular stents, wound drain tubes, hydrocephalus shunts, ventricular catheters, peritoneal catheters, pacemaker systems, small or temporary joint replacements, heart valves, cardiac assist devices and the like and bone prosthesis, joint prosthesis and dental prosthesis.

As regards veterinary medicine, otic maladies (e.g., chronic ear infections) are a common occurrence in animals of veterinary medicine. Otic maladies may result from bacteria associated with otic maladies. Current treatment includes many rounds of antibiotics resulting many times in a reoccurring resistant infection. In some embodiments, compositions described herein may assist in treating the resistant infection (removing the biofilm) making resistant bacteria once again vulnerable to antibiotics.

In some embodiments, compositions described herein may be used to treat otic maladies in the form of an ear cleanser, ear wash, and/or ear drops. In some embodiments, a composition (e.g., otic composition) may include glycerin, propylene glycol, polyethylene, mineral oil, benzyl alcohol, and/or ethyl alcohol. A composition may include a fluid with a high boiling point (e.g., an oil, mineral oil) which may function to spread active ingredients over a surface.

In some embodiments, compositions described herein may be applied topically before, during, and/or after a surgical procedure (e.g., at or around a surgical site or a suture site to inhibit secondary infections).

As regards the petroleum industry, pipeline corrosion and flow restriction is a significant problem caused by biofilms. It is estimated that 40% of the corrosion occurring in the interior of gas pipelines is caused by microorganisms (MIC—microbiologically influenced corrosion), typically sulfate-reducing bacteria. Although some pipelines may be cleaned using mechanical scrubbers others may have to be removed and disassembled to be cleaned. It would be a financial advantage to have a non-corrosive solution that could safely remove biofilms from corroded pipelines. In some embodiments, compositions described herein may assist in treating the corroded pipelines (removing the biofilm).

As regards the food industry, currently used cleaning agents may not adequately remove biofilms from machinery used during food production. Foodborne diseases are a constant threat to human health. Foodborne diseases are considered an emergent public health concern throughout the world. Many outbreaks of foodborne diseases have been found to be associated with biofilm. It is well documented that biofilm has become a problem in food industries as it renders its inhabitants resistant to antimicrobial agents and cleaning.

### EXAMPLES

Having now described the invention, the same will be more readily understood through reference to the following example(s), which are provided by way of illustration, and are not intended to be limiting of the present invention.

Biofilms from a nearby river were selected to serve as a model for the realistic structure of a biofilm. Biofilms are described on the surface of small (15 mm×15 mm) stone.

Biofilms were exposed to various concentrations of Boric Acid. The concentrations used were 0%, 0.25%, 0.5%, 1%, 2%, 3%.

One stone was used per concentration and was measured and weighed for similarity. Each stone was rinsed with sterile H₂O to remove any planktonic (not attached to biofilm) bacteria. Each stone was exposed to a known concentration for a time period of 5 minutes and then removed and placed into a separate vial containing 9 ml of sterile water. The vial with after treatment stone in sterile water was then sonicated for 60 seconds to remove any remaining biofilm to be quantified. Sonication served as a mechanical removal for any remaining biofilm.

To quantify how much biofilm was removed with boric acid, concentrations vs. sonication, each vial was then serial diluted and plated on R2A media. Media was then placed in 30 C for 48 hrs and growth was quantified through counting colonies.

Results showed that a 0.5% (w/v) concentration produced the highest percent release of biofilm. We estimate that the percent release increases with time.

Real Time Visualization of Biofilm Dispersion:

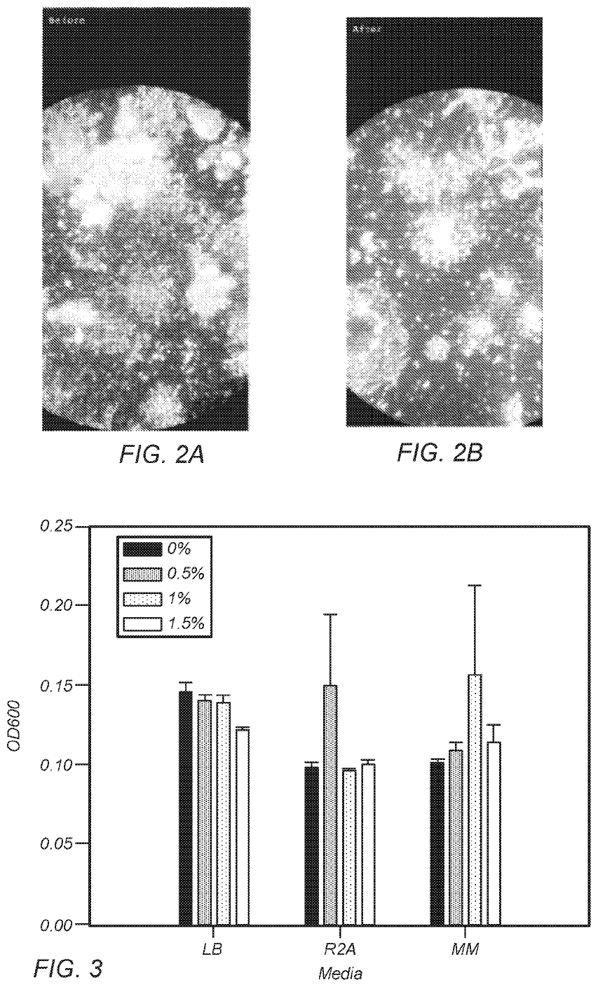

To visualize biofilm dispersion a water sample from the sampling site was collected. A microscope slide was placed in 30 ml of river water to cultivate a biofilm. The biofilm was then viewed under a dark field microscope and a 0.5% concentration of boric acid was applied and left for 60 seconds. FIG. 2A depicts a photograph of a microscope slide of river water with biofilms. FIG. 2B depicts a photograph of a microscope slide of river water with biofilms dispersed after application of a boric acid solution.

Test of application: A 0.5% boric acid was injected into a bioreactor containing a monoculture biofilm of Chromobacterium violaceum. The bioreactor was left for 2 hours and lightly shaken. Results: All visible biofilm was removed from the bioreactor.

Pseudomonas aeruginosa, a common biofilm-associated organism, was used to investigate whether various concentrations of boric acid had an impact on bacterial growth, viability, and whether it could enhance the usefulness of commonly used disinfectants and antibiotics. In most experiments, cultures were grown in 96-well polystyrene microtiter plates as this allowed different growth media and replicates to be tested using an automated plate reader. Growth of suspended (planktonic) bacteria could be measured by the turbidity in the individual microtiter plate chambers (referred to as wells). The biofilms attached to the sides of the microtiter wells. To test for dispersion, the planktonic populations and growth media were removed and replaced with water containing various concentrations of the test compound. Dispersion was observed by an increase in turbidity (due to release of bacteria from biofilms) over 20 minutes. The effectiveness of boric acid supplementation on biofilm treatments was tested in a similar fashion.

P. aeruginosa was grown as planktonic and biofilm populations in different growth media for 48-120 h. Antimicrobial agents were tested in different boric acid concentrations. Boric acid controls lacked the antimicrobial compounds. Initial planktonic growth and dispersion were measured as described previously. After dispersion, the disinfectant and boric acid compounds were removed, replaced with sterile growth medium, and growth monitored by being placed into the plate reader and monitoring an increase of turbidity over an 18-36 h period. Preliminary results suggest a low level of boric acid-induced toxicity at the concentrations listed. In some embodiments, boric acid appears to work best with biofilms grown in low nutrient conditions, rather than in the presence of rich media.

Experiments investigated the ability of P. aeruginosa PAO-1 to grow in the presence of boric acid. Experiments demonstrated that P. aeruginosa grew in the presence of boric acid. Experiments included uninoculated controls; Luria Broth containing 0%, 0.5%, 1.0%, and 1.5% boric acid; R2A broth containing increasing amounts (0, 0.5, 1, and 1.5%) boric acid; and Davis minimal media with the same increase of boric acid. Numerical data showed that bacterial growth of other organisms began to be inhibited at 1.5% (w/v) and higher concentrations of boric acid (e.g., as depicted in FIG. 3).

Experiments were conducted in order to examine the ability of P. aeruginosa biofilms grown on different growth media to be dispersed by boric acid. Here, bacteria were grown for 24 h at 37 C on an orbital shaking plate in a microtiter plate. The media was removed and the adherent P. aeruginosa biofilms were exposed to boric acid, placed in a plate reader and turbidity measured following agitation. The increased turbidity (evidence of dispersion) was seen at 0.5% boric acid for R2A-grown biofilms, and at 1.0% boric acid for Davis Minimal Media-grown biofilms. There was no observable dispersion for LB-grown biofilms, suggesting, in some embodiments, that P. aeruginosa biofilms grown in nutrient-rich media, are not amenable to boric acid-induced dispersion.

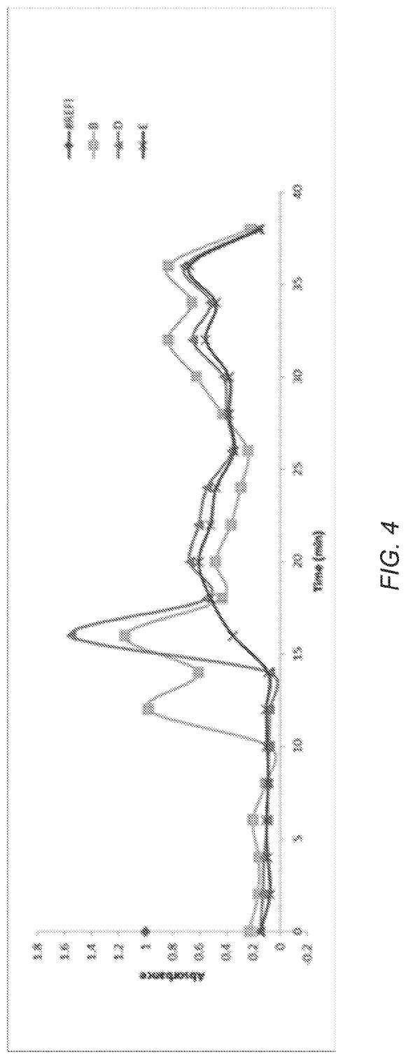

To further study the mechanisms of boric acid-induced dispersion a collaboration with Dr. Karin Sauer, Binghamton University was initiated. Dr. Karin Sauer verified the ability of boric acid to disperse P. aeruginosa biofilms in a flow cell apparatus. FIG. 4 depicts a bar graph summarizing the results of experiments directed towards determining the ability of boric acid to disperse P. aeruginosa biofilms. The line designated as B is boric acid, D represents a positive control for dispersion and E a negative control.

Experiments were conducted in order to determine the ability of boric acid to enhance the antimicrobial action of betadine (poly(vinyl pyrrolidone) iodine). Betadine is commonly used as a disinfectant in clinical environments and the low concentration used (0.02% (w/v)) was chosen based on previous work as it has partial biofilm effectiveness. P. aeruginosa biofilms were grown for 2 and 5 days suspended above a shaking water bath. This allowed precise control of temperature, but there was considerable condensation on the microtiter plate covers which interfered with the spectrophotometer readings. There was no obvious contamination in the uninoculated wells. The media used consisted of artificial urine and artificial urine supplemented with 2 mM glucose (reflecting average urine chemistry in normal and diabetic patients). Davis minimal media (MM) and Luria-Bertani broth (LB) were used as representative minimal and rich media. Different biofilm incubation times were used as it is known that biofilm susceptibility to dispersion can increase with biofilm age.

The testing protocol involved the use of a 96-well microtiter plate and a programmable plate reader (Biotek Instruments). The plate reader was programmed for incubation at 37 C and would agitate the plate for 15 seconds prior to taking an absorbance reading of each well. Microtiter wells were partially filled (200 μl) with sterile growth media, then were inoculated with 2 μl of an overnight bacterial culture suspension. For each media and disinfection combination there were a minimum of two replicates as well as uninoculated wells (media without bacteria) to monitor contamination. The microtiter plates were covered with a lid and the sides sealed with Parafilm™ to prevent contamination. Plates were placed on a rack (above the water level) in a water bath, and incubated with shaking (150 rpm) for 2 and 5 days. Testing consisted of removing a plate from the incubator, placing in the plate reader for 30 minutes (allowing for evaporation of humidity) and reading the turbidity (evidence of planktonic growth). Culture media and planktonic cells were removed from the wells and 200-μl dispersant solution containing a mixture of boric acid and 0.02% (w/v) betadine suspended in PBS. Betadine was absent in the control samples. The microtiter plate was placed in the plate reader for 30 minutes (allowing for evaporation of humidity) and turbidity measurements taken (increased turbidity was evidence of biofilm dispersion). To measure biofilm survival, the plates were removed from the plate reader, the dispersant liquid removed, and replaced with sterile LB growth media. The plates were then placed in the plate reader and growth measured (increased turbidity) over a minimum 18 h period.

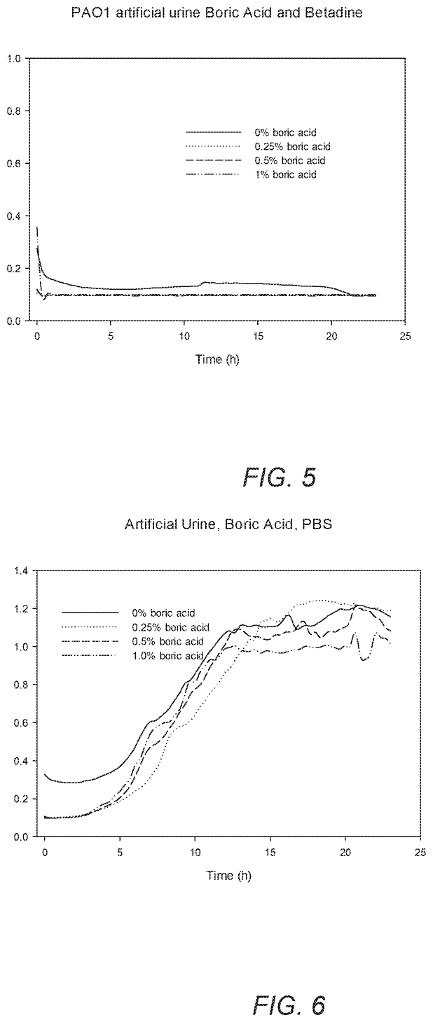

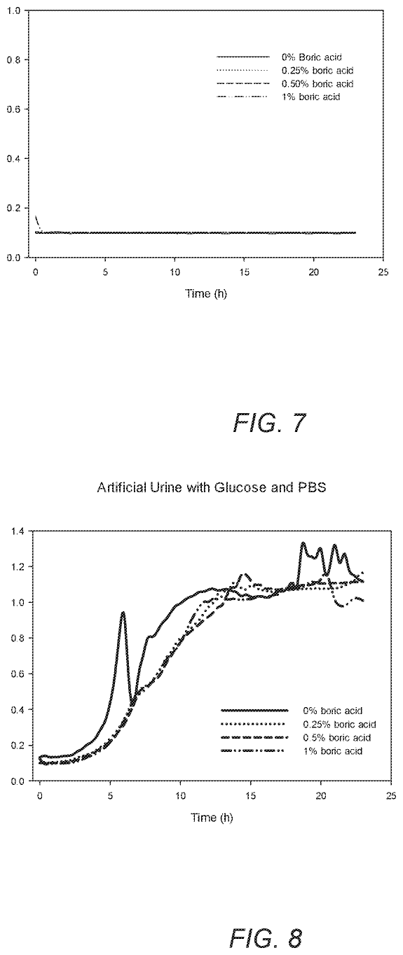

Of note in FIG. 5 (boric acid suspended in phosphate buffered saline (PBS), plus 0.02% betadine), there was no increase in turbidity (interpreted as growth) beyond 1 hour in samples containing boric acid. The turbidity prior to that time is due to condensation on the lid of the microtiter plate. Those two results are for 2 day old biofilms. In FIG. 6, growth did occur when betadine was not used, suggesting that the combination of betadine and boric acid is effective. FIG. 7 indicates artificial urine with glucose (i.e., diabetic artificial urine). There is no growth observed with betadine, although in that case boric acid had no effect. In the control graph depicted in FIG. 8, the data variability comes from condensation on the chamber surface.

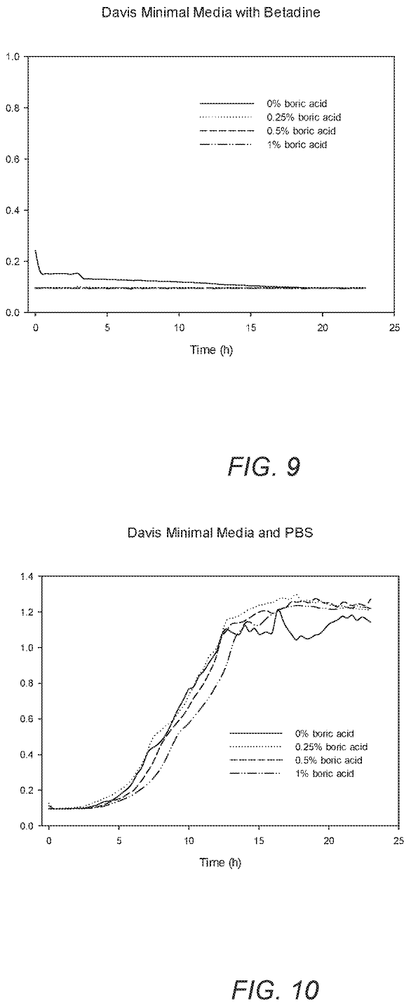

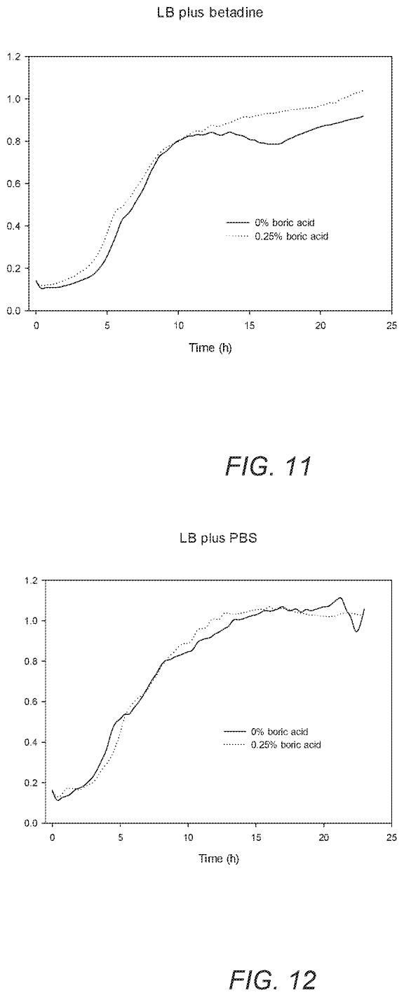

The two-day biofilm detachment studies show that betadine activity against biofilms is enhanced in the presence of boric acid (lack of an increase in turbidity (indicating no growth) as depicted in FIGS. 9 and 11). However boric acid treatment alone did not kill P. aeruginosa as indicated by the graphs in FIGS. 10 and 12.

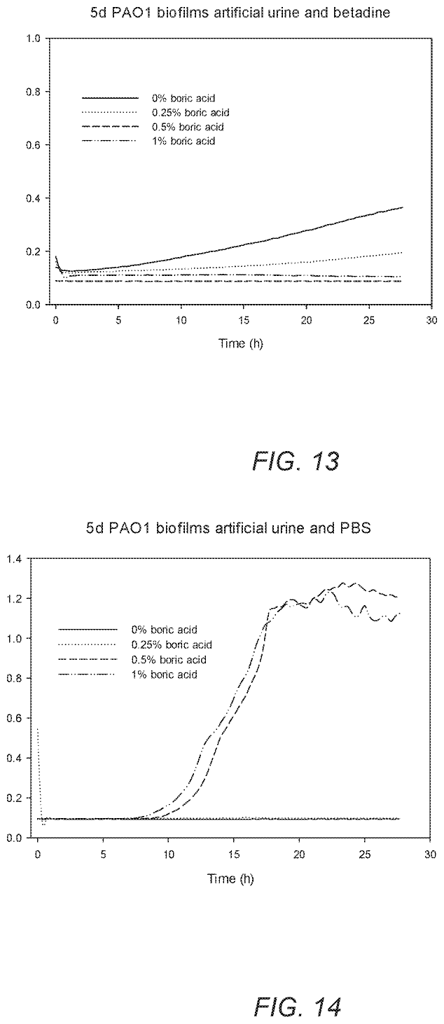

FIGS. 13 and 14 depict results for 5-day old Pseudomonas aeruginosa PAO-1 biofilms grown in artificial urine with the betadine-boric acid treatment in FIG. 13 and the control (boric acid-PBS) is depicted in FIG. 14. However, these preliminary results suggest that boric acid does enhance the activity of betadine against older P. aeruginosa biofilms.

In this patent, certain U.S. patents, U.S. patent applications, and other materials (e.g., articles) have been incorporated by reference. The text of such U.S. patents, U.S. patent applications, and other materials is, however, only incorporated by reference to the extent that no conflict exists between such text and the other statements and drawings set forth herein. In the event of such conflict, then any such conflicting text in such incorporated by reference U.S. patents, U.S. patent applications, and other materials is specifically not incorporated by reference in this patent.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as the presently preferred embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

What is claimed is:

1. A method of inhibiting and/or dispersing a biofilm, comprising:
administering a composition to a biofilm on a surface, wherein the composition comprises a boric acid; and
dispersing the biofilm using the composition.

2. The method of claim 1, wherein the biofilm comprises a plurality of microorganisms coupled together.

3. The method of claim 1, further comprising dispersing the biofilm by uncoupling at least some of the plurality of microorganisms.

4. The method of claim 1, wherein boric acid comprises H₃BO₃.

5. The method of claim 1, wherein boric acid comprises sodium borate

6. The method of claim 1, wherein at least a portion of the composition is dissolved in a solvent, and wherein the boric acid has a 0.5% (w/v) concentration.

7. The method of claim 1, wherein at least a portion of the composition is dissolved in a solvent, and wherein the boric acid has a 0.4% to 1.1% or 0.5% to 1.0% (w/v) concentration.

8. The method of claim 1, wherein at least a portion of the composition is dissolved in a solvent, and wherein the boric acid has about a 0.1% to about a 5% (w/v) concentration.

9. The method of claim 1, wherein at least a portion of the composition is dissolved in a solvent, and wherein the solvent comprises water.

10. The method of claim 1, further comprising administering to the surface an antimicrobial treatment.

11. The method of claim 1, further comprising administering to the surface an antimicrobial treatment, wherein the antimicrobial treatment comprises at least one of biocides, surfactants, antibiotics, antiseptics, detergents, chelating agents, virulence factor inhibitors, ultrasonic treatment, radiation treatment, thermal treatment, and mechanical treatment.

12. The method of 1, wherein the surface comprises at least a portion of a maritime vessel.

13. The method of claim 1, wherein the surface comprises at least a portion of a medical device.

14. The method of claim 1, wherein the surface comprises at least a portion of an otic surface of an animal.

15. The method of claim 1, wherein the surface comprises at least a portion of a petroleum industrial pipeline.

16. The method of claim 1, wherein the surface comprises at least a portion of equipment associated with the food industry.

17. The method of claim 1, wherein the biofilm is formed by Proteobacteria.

18. The method of claim 1, wherein the biofilm is formed by Gram-negative bacteria.

19. The method of claim 1, wherein the biofilm is formed by Firmicutes.

20. The method of claim 1, wherein the biofilm is formed by Gram-positive bacteria.

21. A method of inhibiting and/or dispersing a biofilm, comprising:
administering a composition to a biofilm on a surface, wherein the composition comprises a boric acid, and wherein at least a portion of the composition is dissolved in a solvent;
dispersing the biofilm using the composition; and
administering to the surface an antimicrobial treatment.

22. The method of claim 21, wherein the biofilm comprises a plurality of microorganisms coupled together.

23. The method of claim 21, further comprising dispersing the biofilm by uncoupling at least some of the plurality of microorganisms.

24. The method of claim 21, wherein boric acid comprises H₃BO₃.

25. The method of claim 21, wherein the boric acid has a 0.5% (w/v) concentration.

26. The method of claim 21, wherein the boric acid has about a 0.1% to about a 5% (w/v) concentration.

27. The method of claim 21, wherein the solvent comprises water.

28. The method of claim 21, wherein the antimicrobial treatment comprises at least one of biocides, surfactants, antibiotics, antiseptics, detergents, chelating agents, virulence factor inhibitors, ultrasonic treatment, radiation treatment, thermal treatment, and mechanical treatment.

29. A method of inhibiting and/or dispersing a biofilm, comprising:
administering a composition to a biofilm on a surface, wherein the composition comprises a boric acid; and
dispersing the biofilm using the composition.

30. A composition, comprising:
boric acid; and
a solvent configured to dissolve at least a portion of the boric acid;
wherein the composition disperses a biofilm on a surface.

31. A method of inhibiting formation of a biofilm, comprising:
administering a composition to a surface, wherein the composition comprises a boric acid; and
inhibiting formation of a biofilm on the surface using the composition.

32. A method of inhibiting and/or dispersing a biofilm, comprising:
administering a composition to a biofilm on a surface, wherein the composition comprises a boric acid.
